(19) United States
(12) Patent Application Publication
(10) Pub. No.: US 2020/0309734 A1
(43) Pub. Date: Oct. 1, 2020

(54) SENSOR ELEMENT AND GAS SENSOR

(71) Applicant: NGK INSULATORS, LTD., Nagoya, (JP)

(72) Inventors: Mika KAI, Nagoya (JP); Ryo HAYASE, Nagoya (JP)

(21) Appl. No.: 16/829,060

(22) Filed: Mar. 25, 2020

(30) Foreign Application Priority Data
Mar. 28, 2019 (JP) 2019-064032

(51) Int. Cl. G01N 27/407; G01N 33/00

(52) U.S. Cl. CPC G01N 27/4077; G01N 27/4074; G01N 33/0037

(57) ABSTRACT

A sensor element includes an elongated element body that includes an oxygen-ion-conductive solid electrolyte layer, the element body being in a rectangular parallelepiped shape having a longitudinal direction; a detection unit that has a plurality of electrodes disposed on a front-end side of the element body, and detects a specific gas concentration in a measurement-object gas, the longitudinal direction being a front-rear direction; an outside electrode that is one of the plurality of electrodes and disposed on a first face which is a surface along the longitudinal direction of the element body; and a porous first protective layer that is disposed on the first face and covers the outside electrode. A surface area S of a rear end face of the first protective layer is greater than or equal to 0.9 mm².

This application claims priority based on Japanese Patent Application No. 2019-064032 filed on Mar. 28, 2019, the entire contents of which are incorporated herein by reference.

### BACKGROUND OF THE INVENTION

### 1. Field of the Invention

The present invention relates to a sensor element and a gas sensor.

### 2. Description of the Related Art

A conventional gas sensor is known which includes a sensor element that detects a concentration of a specific gas such as NOx in a measurement-object gas, such as exhaust gas of automobiles. In addition, it is known that a porous protective layer is formed on the surface of the gas device (for instance, PTL 1, PTL 2). PTL 1, PTL 2 describe a sensor element that includes an elongated element body in a rectangular parallelepiped shape, and a porous protective layer that covers the surface of the element body on the front-end side and also an outside electrode disposed outside of the element body. PTL 1 describes that the porous protective layer can be formed by the dipping method, the screen printing, the gel casting method, or the plasma spraying and the like. PTL 2 describes a method of forming a porous protective layer by the plasma spraying using a plasma gun and a mask.

### CITATION LIST

### Patent Literature

PTL 1: Japanese Unexamined Patent Application Publication No. 2017-187482

PTL 2: Japanese Unexamined Patent Application Publication No. 2016-109685

### SUMMARY OF THE INVENTION

Meanwhile, there is a case where water in a measurement-object gas is condensed and adheres to an element body during use of a sensor element, then the water moves along the element body and adheres to the rear end of a protective layer. The water may contain poisonous substances (for instance, P, Si, S, Mg and the like) in the measurement-object gas. Thus, when the water adhering to the rear end of the protective layer reaches the outside electrode through the protective layer, the poisonous substances may adhere to the outside electrode, and abnormality, such as reduction in the accuracy of detection of a specific gas concentration by the sensor element, may occur.

The present invention has been devised to solve such a problem, and the main object is to inhibit the water containing poisonous substances from reaching the outside electrode.

The present invention adopts the following device to achieve the main object.

The sensor element of the present invention comprises: an element body that includes an oxygen-ion-conductive solid electrolyte layer, the element body being in an elongated rectangular parallelepiped shape having a longitudinal direction;

a detection unit that has a plurality of electrodes disposed on a front-end side of the element body, and detects a specific gas concentration in a measurement-object gas, the longitudinal direction being a front-rear direction;

an outside electrode that is one of the plurality of electrodes and disposed on a first face which is a surface along the longitudinal direction of the element body; and

a porous first protective layer that is disposed on the first face and covers the outside electrode.

A surface area S of a rear end face of the first protective layer is greater than or equal to 0.9 mm².

In the sensor element, the first porous protective layer covers the outside electrode. The surface area S of the rear end face of the first protective layer is greater than or equal to 0.9 mm². Since the surface area S is large like this, when the water, which moves forward along the surface of the element body, adheres to the rear end face of the first protective layer, the water is likely to spread in a surface direction on the rear end face. Therefore, it is possible to inhibit the water adhering to the rear end face and containing poisonous substances from reaching the outside electrode through the inside of the first protective layer.

In the sensor element of the present invention, a minimum distance D from the rear end of the outside electrode to a contact portion between the rear end face and the first face may be greater than or equal to 2 mm. Since the minimum distance D is large like this, the outside electrode is present at a position away from the contact portion between the rear end face and the first face, in other words, the portion at which water moving forward along the first face of the element body reaches the rear end face for the first time. Thus, with the minimum distance D greater than or equal to 2 mm, it is possible to further inhibit the water adhering to the rear end face from reaching the outside electrode through the inside of the first protective layer.

In the sensor element of the present invention, a thickness T of the first protective layer may be greater than or equal to 0.03 mm and less than or equal to 1 mm. With the thickness T greater than or equal to 0.03 mm, it is possible to inhibit the water adhering to the surface of the first protective layer and containing poisonous substances from moving through the inside of the first protective layer in a thickness direction and reaching the outside electrode. In addition, with the thickness T less than or equal to 1 mm, it is possible to inhibit the reduction in the responsiveness of detection of a specific gas concentration by the sensor element.

In the sensor element of the present invention, the rear end face of the first protective layer may have a shape curved to be recessed at a center between right and left, and the direction of the right and left is parallel to the first face and perpendicular to the longitudinal direction. In this manner, the surface area S is likely to be increased, as compared with when the rear end face has a planar shape without a curve.

In the sensor element of the present invention, the rear end face of the first protective layer may have an inclination angle θ of 10° or greater and 90° or less with respect to the first face.

The gas sensor of the present invention includes the sensor element in one of the aspects described above. Thus, the gas sensor provides the same effect as that of the above-described sensor element of the present invention, for instance, the effect of inhibiting the water containing poisonous substances from reaching the outside electrode.

### BRIEF DESCRIPTION OF THE DRAWINGS

FIG. 1 is a vertical cross-sectional view of a gas sensor 100.

FIG. 2 is a perspective view schematically illustrating an example of a configuration of a sensor element 10.

FIG. 3 is a cross-sectional view taken along A-A of FIG. 2.

FIG. 4 is a vertical cross-sectional view of a rear end face 31a of a first protective layer 31.

FIG. 5 is an explanatory diagram illustrating the manner in which a surface area S is derived.

FIG. 6 is an explanatory diagram illustrating the manner in which a surface area S is derived.

FIG. 7 is a top view illustrating a rear end face 31a in a modification.

FIG. 8 is a top view illustrating a rear end face 31a in a modification.

FIG. 9 is a top view illustrating a rear end face 31a in a modification.

FIG. 10 is a vertical cross-sectional view illustrating a rear end face 31a in a modification.

FIG. 11 is a vertical cross-sectional view illustrating a porous protective layer 30 in a modification.

FIG. 12 is a vertical cross-sectional view illustrating a porous protective layer 30 in a modification.

FIG. 13 is a top view illustrating a rear end face 31a in a modification.

FIG. 14 is an explanatory view illustrating the manner in which a poisoning resistance test is conducted.

### DETAILED DESCRIPTION OF THE INVENTION

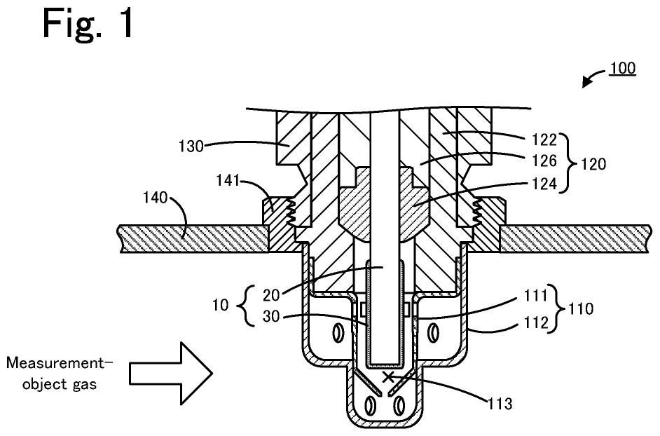

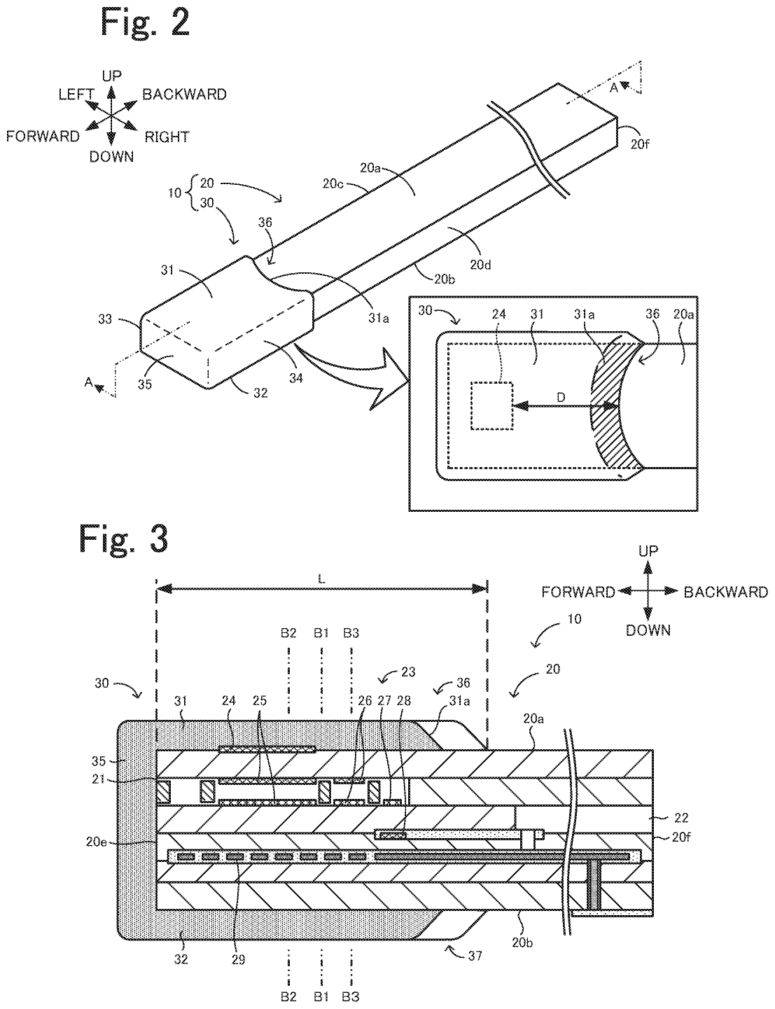

Next, an embodiment of the present invention will be described using the drawings. FIG. 1 is a vertical cross-sectional view of a gas sensor 100 which is an embodiment of the present invention. FIG. 2 is a perspective view schematically illustrating an example of a configuration of a sensor element 10. FIG. 3 is a cross-sectional view taken along A-A of FIG. 2. The configuration of the gas sensor 100 as illustrated in FIG. 1 is publicly known, and is described, for instance, in Japanese Unexamined Patent Application Publication No. 2012-210637.

The gas sensor 100 includes a sensor element 10; a protective cover 110 that covers one end (the lower end of FIG. 1) of the sensor element 10 in the longitudinal direction; a device sealing body 120 that seals and fixes the sensor element 10; and a nut 130 mounted on the device sealing body 120. As illustrated, the gas sensor 100 is mounted on, for instance, a pipe 140 such as an exhaust gas pipe and used to measure the concentration of a specific gas (NOx in the present embodiment) which is contained in exhaust gas as a measurement-object gas.

The protective cover 110 includes a bottomed tubular inside protective cover 111 that covers one end of the sensor element 10; and a bottomed tubular outside protective cover 112 that covers the inside protective cover 111. A plurality of holes for distributing a measurement-object gas into the protective cover 110 are formed in the inside protective cover 111 and the outside protective cover 112. A device chamber 113 is formed as the space surrounded by the inside protective cover 111, and the front end of the sensor element 10 is disposed in the device chamber 113.

The device sealing body 120 includes a cylindrical main metal fitting 122; a ceramic supporter 124 sealed in a through hole inside the main metal fitting 122; and a powder compact 126 which is obtained by molding ceramic powder, such as talc, sealed in a through hole inside the main metal fitting 122. The sensor element 10 is positioned on the central axis of the device sealing body 120, and penetrates the device sealing body 120 in a front-rear direction. The powder compact 126 is compressed between the main metal fitting 122 and the sensor element 10. Thus, the powder compact 126 seals the through hole in the main metal fitting 122 and fixes the sensor element 10.

The nut 130 is fixed concentrically to the main metal fitting 122, and has an outer circumferential surface with a male screw portion formed thereon. The male screw portion of the nut 130 is inserted in a mounting member 141 which is welded to the pipe 140 and has an inner circumferential surface with a female screw portion provided thereon. Thus, the gas sensor 100 can be fixed to the pipe 140 with one end of the sensor element 10 and a portion of the protective cover 110 projecting into the pipe 140.

As illustrated in FIGS. 2, 3, the sensor element 10 includes an element body 20, a detection unit 23, a heater 29, and a porous protective layer 30. As illustrated in FIGS. 2 and 3, the element body 20 has an elongated rectangular parallelepiped shape. The longitudinal direction of the element body 20 is the front-rear direction, the thickness direction of the element body 20 is the up-down direction, and the width direction of the element body 20 is the right-left direction.

The element body 20 has a laminated body in which a plurality of (six in FIG. 3) layers of oxygen-ion-conductive solid electrolyte such as zirconia (ZrO₂) are laminated in the thickness direction. Since the element body 20 has a rectangular parallelepiped shape, as illustrated in FIGS. 2, 3, the element body 20 has first to sixth faces 20a to 20f as the outer faces. The first and second faces 20a, 20b are the faces located at both ends of the element body 20 in the thickness direction. The third and fourth faces 20c, 20d are the faces located at both ends of the element body 20 in the width direction. The fifth and sixth faces 20e, 20f are the faces located at both ends of the element body 20 in the length direction. The first face 20a is a surface along the longitudinal direction of the element body 20, and is the top face of the element body 20. The dimensions of the element body 20 may be, for instance, a length of 25 mm or greater and 100 mm or less, a width of 2 mm or greater and 10 mm or less, and a thickness of 0.5 mm or greater and 5 mm or less. In addition, a gas introduction port 21, and a reference gas introduction port 22 are formed in the element body 20, the gas introduction port 21 being open to the fifth face 20e for introducing a measurement-object gas to the inside of the element body 20, the reference gas introduction port 22 being open to the sixth face 20f for introducing a reference gas (air herein) serving as a reference to detect a specific gas concentration to the inside of the element body 20. In the element body 20, space is provided from the gas introduction port 21 to a measurement electrode 27, and the space is referred to as the measurement-object gas distribution portion.

The detection unit 23 is for detecting a specific gas concentration in a measurement-object gas. The detection unit 23 has a plurality of electrodes 23 disposed on the front-end side of the element body 20. In the present embodiment, the detection unit 23 includes an outside electrode 24, an inside main pump electrode 25, an inside auxiliary pump electrode 26, a measurement electrode 27, and a reference electrode 28 as the plurality of electrodes. The outside electrode 24 is disposed on the first face 20a. The inside main pump electrode 25, the inside auxiliary pump electrode 26, and the measurement electrode 27 are disposed in the inside of the element body 20, and are disposed in that order in the measurement-object gas distribution portion from the gas introduction port 21 toward the rear. The reference electrode 28 is disposed in the inside of the element body 20, and a reference gas reaches the reference electrode 28 through the reference gas introduction port 22. The inside main pump electrode 25 and the inside auxiliary pump electrode 26 are disposed on the inner circumferential surface of the space inside the element body 20, and may have a tunnel-like structure.

The outside electrode 24 is formed as, for instance, a porous cermet electrode (for instance, a cermet electrode composed of Au and Pt, and ZrO₂). Similarly, other electrodes 25 to 28 included in the detection unit 23 may be formed as porous cermet electrodes.

Since the principle for detecting a specific gas concentration in a measurement-object gas using the detection unit 23 is well known, a detailed description is omitted. The detection unit 23 operates, for instance, in the following manner. The detection unit 23 pumps out and pumps in the oxygen in a measurement-object gas in the vicinity of the inside main pump electrode 25 to and from the outside (the device chamber 113) based on a voltage applied across the outside electrode 24 and the inside main pump electrode 25. In addition, the detection unit 23 pumps out and pumps in the oxygen in the measurement-object gas in the vicinity of the inside auxiliary pump electrode 26 to and from the outside (the device chamber 113) based on a voltage applied across the outside electrode 24 and the inside auxiliary pump electrode 26. Thus, the measurement-object gas with an oxygen concentration adjusted to a predetermined value reaches the vicinity of the measurement electrode 27. The measurement electrode 27 functions as a NOx reduction catalyst, and reduces the specific gas (NOx) in the reached measurement-object gas. The detection unit 23 then pumps out the oxygen in the measurement-object gas in the vicinity of the measurement electrode 27 to the outside (the device chamber 113) based on a voltage applied across the outside electrode 24 and the measurement electrode 27. Thus, the detection unit 23 pumps out the oxygen in the vicinity of the measurement electrode 27 to the outside so that the oxygen generated by reducing NOx in the measurement-object gas becomes substantially zero. At this point, a pump current Ip2 flows between the outside electrode 24 and the measurement electrode 27. The pump current Ip2 takes a value (a value by which a specific gas concentration is derivable) according to the specific gas concentration in the measurement-object gas.

The heater 29 is an electrical resistance element disposed inside the element body 20. The heater 29 generates heat by power feed from the outside, and heats the element body 20. The heater 29 can adjust the temperature of the solid electrolyte layer to a level (for instance, 800° C.) at which the solid electrolyte layer is activated, by heating and maintaining the temperature of the solid electrolyte layer of the element body 20.

The porous protective layer 30 is a porous body that covers the front-end side surface of the element body 20, particularly, the portion of the element body 20, positioned in the device chamber 113. In the present embodiment, the porous protective layer 30 includes first to fifth protective layers 31 to 35 respectively disposed on five faces (the first to fifth faces 20a to 20e) out of the six faces of the element body 20. The first protective layer 31 covers part of the top face of the element body 20, that is, the first face 20a. Similarly, the second to fourth protective layers 32 to 34 cover part of the bottom face (the second face 20b), the left face (the third face 20c), and the right face (the fourth face 20d), respectively. The fifth protective layer 35 covers the front face of the element body 20, that is, the entire fifth face 20e. In the first to fifth protective layers 31 to 35, adjacent layers are connected, and the entire porous protective layer 30 covers the front-end face (the fifth face 20e) and its surroundings.

The first protective layer 31 indicates a portion of the porous protective layer 30, the portion being present immediately above the first face 20a. Thus, in the present embodiment, the front-rear length of the first protective layer 31 is equal to a distance L (see FIG. 3) from the front end of the first face 20a to the rear end of the first protective layer 31 in the front-rear direction. Also, in the present embodiment, as seen from the enlarged top view of the first protective layer 31 and its vicinity, illustrated at the lower right of FIG. 2, the width of the first protective layer 31 is equal to the width of the first face 20a. The first protective layer 31 also covers the outside electrode 24 disposed on the first face 20a. A recessed portion 36 is formed at the rear-end side of the first protective layer 31 of the porous protective layer 30. The recessed portion 36 has a shape curved to be recessed deeper at the center between the right and left of the first protective layer 31. The first protective layer 31 has a rear end face 31a, and the rear end face 31a constitutes part of the recessed portion 36. Thus, the rear end face 31a has a shape curved to be recessed deeper at the center between the right and left. As illustrated by hatching in FIG. 2, the rear end face 31a of the first protective layer 31 indicates only the portion immediately above the first face 20a. The recessed portion 36 is formed on the outer sides of the first face 20a along the right and left, in other words, part of the third and fourth protective layers 33, 34 also constitute the recessed portion 36. However, the recessed portion 36 may be formed only on the portion immediately above the first face 20a, that is, the recessed portion 36 may match the rear end face 31a.

The porous protective layer 30 is formed symmetrically in the up-down direction and symmetrically in the right-left direction in the present embodiment. Thus, a recessed portion 37 similar to the recessed portion 36 is formed on the rear-end side of the second protective layer 32 of the porous protective layer 30 (see FIG. 3). Also, the front-rear length of each of the second to fourth protective layers 32 to 34 is equal to the distance L. Although the fifth protective layer 35 also covers the gas introduction port 21, a measurement-object gas can reach the gas introduction port 21 by flowing through the inside of the fifth protective layer 35 because the fifth protective layer 35 is a porous body.

The porous protective layer 30 covers the front-end side of the element body 20, and protects the portion. The porous protective layer 30 achieves the function of inhibiting the water or the like in a measurement-object gas from adhering to the element body 20 and causing a crack therein. In addition, the first protective layer 31 achieves the function of inhibiting poisonous substances (for instance, P, Si, S, Mg and the like) contained in the measurement-object gas from adhering to the outside electrode 24 and inhibiting degradation of the outside electrode 24. The distance L is determined in the range of (0<the distance L<the length of the element body 20 in the longitudinal direction) based on the range in which the element body 20 is exposed to the measurement-object gas in the gas sensor 100, and the position of the outside electrode 24.

The porous protective layer 30 is, for instance, a porous body of ceramics. It is preferable that the porous protective layer 30 include particles of at least one of alumina, zirconia, spinel, cordierite, titania, and magnesia. In the present embodiment, the porous protective layer 30 is ceramics containing alumina as the main component.

The degree of porosity of the first protective layer 31 may be, for instance, 10% or greater and 60% or less, or 10% or greater and 40% or less. The degree of porosity is the value measured based on the mercury intrusion technique in conformity with JIS R1655. An arithmetic average roughness Ra of the top face of the first protective layer 31 and the rear end face 31a may be, for instance, 2 μm or greater and 30 μm or less.

The rear end face 31a of the first protective layer 31 has the surface area S greater than or equal to 0.9 mm². It is preferable that the thickness T of the first protective layer 31 be 0.03 mm or greater and 1 mm or less. The reason why these values are preferable will be described later. Also, the rear end face 31a of the first protective layer 31 may have an inclination angle θ of 10° or greater and 90° or less with respect to the first face 20a. A method of measuring the surface area S, the thickness T, and the inclination angle θ will be described in the following.

A method of measuring the thickness T of the first protective layer 31 will be described. First, CT scan is performed on the sensor element 10, and a cross section (cross section taken along B1-B1 of FIG. 3) at the front-rear center of the first protective layer 31 is captured. The B1-B1 cross section is a cross section perpendicular to the first face 20a and perpendicular to the front-rear direction. Then a maximum value of the thickness in the B1-B1 cross section of the first protective layer 31 is derived from an image obtained. Similarly, an image is captured by CT scan for the B2-B2 cross section and the B3-B3 cross section illustrated in FIG. 3, and a maximum value of the thickness in each cross section is derived. Then the thickness T of the first protective layer 31 is set to the average value of the derived three maximum values.

The surface of the first protective layer 31 normally has fine bumps and dips, thus the thickness T is set to the average value measured by using three cross sections in this manner. Here, the B2-B2 cross section is the cross section positioned forward from the B1-B1 cross section by 3 mm. The B3-B3 cross section is the cross section positioned rearward from the B1-B1 cross section by 3 am. In short, the front-rear interval of the three cross sections is 3 am. However, when the front-rear length (here, the distance L) of the first protective layer 31 is less than 10 mm, the front-rear interval of the three cross sections is not 3 mm, but “the front-rear length of the first protective layer 31×0.3”.

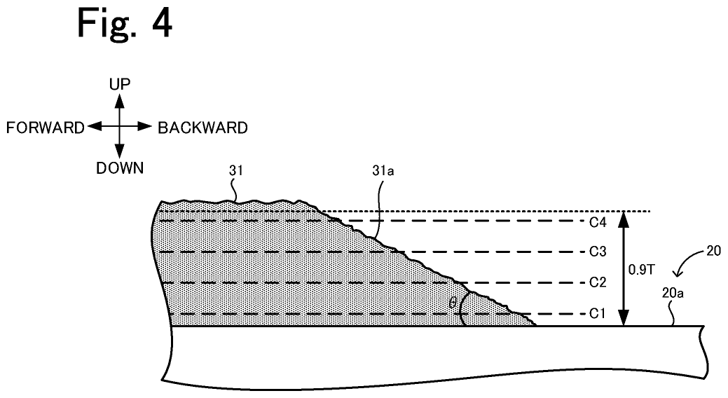

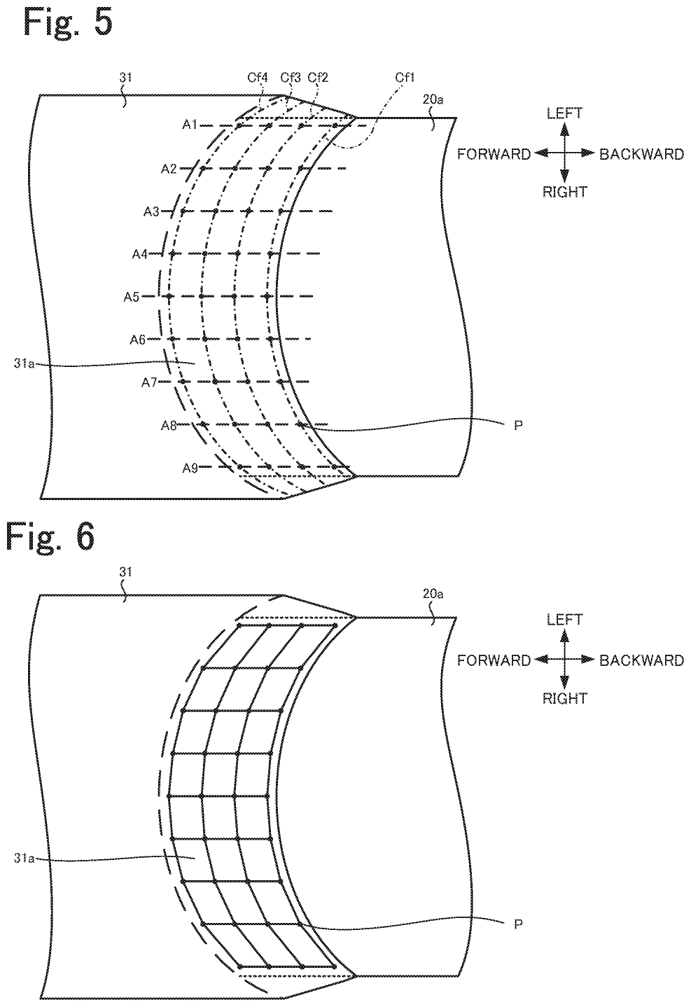

A method of measuring the surface area S of the rear end face 31a will be described. FIG. 4 is a vertical cross-sectional view of the rear end face 31a of the first protective layer 31. FIG. 5 and FIG. 6 are explanatory diagrams illustrating the manner in which the surface area S is derived. FIGS. 5, 6 are top views of the rear end face 31a and its vicinity.

First, CT scan is performed on the rear end face 31a with 15 μm interval in the up-down direction, and m pieces of data of contour (contour parallel to the first face 20a) of the rear end face 31a. At this point, there is a case where it is difficult to clearly define the boundary line between the rear end face 31a and the top face of the first protective layer 31, thus CT scan is performed on the range of the rear end face 31a, where the thickness is up to 0.9 T. Thus, m is the value obtained by adding 1 to the quotient “(0.9× the thickness T)/15 μm”. FIG. 4 illustrates the manner in which m=4 and CT scan for four cross sections C1 to C4 is performed.

Next, filtering, which ignores bumps and dips corresponding to the arithmetic average roughness Ra less than 50 μm, is performed on each of m pieces of data of contour to obtain data of contour with fine bumps and dips removed. The processing to perform such filtering can be conducted using a Gaussian filter, for instance. Contours Cf1 to Cf4 each illustrated by a dashed-dotted line of FIG. 5 are an example of m pieces (four, herein) of data of contour. The contours Cf1 to Cf4 of FIG. 5 show an example of data of contour obtained from the respective cross sections of C1 to C4 of FIG. 4.

Subsequently, for each of the m pieces of data of contour, n representative points P with a 15 μm interval in the right-left direction are determined (see black dots of FIG. 5). The arrangement of the representative points P is made within the range of the width of the rear end face 31a. Thus, n is the value obtained by adding 1 to the quotient “the width of the rear end face 31a (equals the width of the first face 20a herein)/15 μm”. FIG. 5 illustrates the case with n=9. Thus, mm (4×9=36 in FIG. 5) representative points P are determined. That is, for each of the m×n representative points P, the position (coordinates) in three-dimensional space is determined. n representative points P disposed on each of the m contours are determined so that the representative points P are positioned on lines disposed with a 15 μm interval in the right-left direction in a top view. For instance, when n=9, m×n representative points P are determined as the intersection points between dashed lines A1 to A9 (lines disposed with a 15 μm interval in the right-left direction and parallel to the front-rear direction) and m contours (the contours Cf1 to Cf4 herein).

Adjacent representative points P of thus determined mm representative points P are connected with a line, thereby determining a plurality of ((m−1)×(n−1)) quadrilaterals. For instance, when the representative points P are determined as in FIG. 5, 3×8=24 quadrilaterals are determined as illustrated in FIG. 6. Thus, the shape of the rear end face 31a is approximated by a plurality of quadrilaterals which are adjacent to each other. Although the quadrilaterals are illustrated in a top view in FIG. 6, as seen from FIG. 4, the quadrilaterals of FIG. 6 are inclined to reflect the inclination of the rear end face 31a, thus also simulate the inclination of the rear end face 31a.

Thus, the total value of the areas of (m−1)×(n−1) quadrilaterals is derived as an area Sp. However, the area Sp is only part of the rear end face 31a as seen from FIG. 6, thus is converted (extended to) to a value which is regarded as the area of the entire rear end face 31a, and the value is defined as the surface area S of the rear end face 31a. Specifically, the surface area S is derived from the following Expression (1). “(m−1)×15 μm” in Expression (1) shows the height from C1 to C4 in FIG. 4, for instance. The “width of the rear end face 31a” is the same as the width of the first face 20a herein. “(n−1)×15 μm” in Expression (1) shows the width from the left end to the right end of the set union of the quadrilaterals in FIG. 6, for instance, in other words, shows the right-left length from the dashed line A1 to A9 of FIG. 5.

Surface area S=area Sp×{thickness T/((m−1)×15 μm)}×{width of the rear end face 31a/((n−1)×15 μm)} Expression (1)

In this manner, the surface area S is determined as a value approximating the shape of the rear end face 31a using the representative points P and the quadrilaterals. Thus, the value of the surface area S is a value which ignores fine bumps and dips, and reflects the shape of the rear end face 31a even though the shape is curved as illustrated in FIG. 2, or inclined as illustrated in FIGS. 3, 4.

The CT scan for measuring the thickness T and the surface area S described above can be performed using, for instance, SMX-160CT-SV3 manufactured by Shimazu Corporation.

A method of measuring the inclination angle θ of the rear end face 31a will be described using FIG. 5. First, an angle θ1 formed by the first face 20a and the line connecting two points at both ends in the front-rear direction between the m (four herein) representative points P positioned on the dashed line A1 of FIG. 5. Similarly, angles θ2 to θ9 are determined for the m representative points P positioned on the dashed lines A2 to A9. Then let the inclination angle θ be the average value of the determined angles θ1 to θ9. The inclination angle θ (and the formed angles θ1 to θ9) are each an angle on the side including the first protective layer 31 as illustrated in FIG. 4.

Let a minimum distance D be the minimum value of the distance from the rear end of the outside electrode 24 to the contact portion between the rear end face 31a and the first face 20a. The minimum distance D is preferably 2 mm or greater for same reason which will be described later. The minimum distance D is determined as the distance (distance in a direction parallel to the first face 20a) in a top view (when the outside electrode 24 and the first protective layer 31 are viewed perpendicularly to the first face 20a) as illustrated at the lower right of FIG. 2. In the present embodiment, the contact portion between the rear end face 31a and the first face 20a is the curved portion which appears as the contour of the rear end of the rear end face 31a in a top view. Thus, the minimum value of the distance between the curved portion and the rear end of the outside electrode 24 is the minimum distance D.

A method of manufacturing thus configured gas sensor 100 will be described in the following. First, a method of manufacturing the sensor element 10 will be described. When the sensor element 10 is manufactured, multiple pieces (six pieces herein) of unfired ceramic green sheets, corresponding to the element body 20 are prepared. In each green sheet, a notch or a through hole or a groove is provided by a stamping process as needed, and electrodes and a wiring pattern are screen-printed. Subsequently, a plurality of green sheets are laminated, bonded, and fired, thereby producing the sensor element 10. Formation of the porous protective layer 30 may be achieved by forming the first to fifth protective layers 31 to 35 one by one. When the first protective layer 31 is formed, the shape of the rear end face 31a, the surface area S, the inclination angle θ, and the minimum distance D can be adjusted, for instance, by the shape of a mask used for plasma spraying, and the position of the mask on the element body 20. The minimum distance D can also be adjusted by the position of the outside electrode 24. The thickness T can be adjusted, for instance, by the length of spraying time.

Formation of the porous protective layer 30 may be achieved not only by plasma spraying, but also by the dipping method, the screen printing, the gel casting method and the like. Even when the first protective layer 31 is formed by these methods, it is possible to adjust the shape of the rear end face 31a, the surface area S, the inclination angle θ, and the minimum distance D by adjusting, for instance, the shape and position of the mask, and adjusting the viscosity of paste which serves as the porous protective layer 30.

Next, the gas sensor 100, in which the sensor element 10 is incorporated, is produced. First, the sensor element 10 is caused to penetrate the inside of the main metal fitting 122 in the axial direction, and the supporter 124 and the powder compact 126 are disposed between the inner circumferential surface of the main metal fitting 122 and the sensor element 10. Next, the powder compact 126 is compressed, and a portion of the device sealing body 120 is sealed, the portion being between the inner circumferential surface of the main metal fitting 122 and the sensor element 10. Subsequently, the protective cover 110 is welded to the device sealing body 120, and the nut 130 is mounted thereon.

Next, a use example of thus configured gas sensor 100 will be described in the following. When a measurement-object gas flows in the pipe 140 with the gas sensor 100 mounted on the pipe 140 as illustrated in FIG. 1, the measurement-object gas is distributed in the protective cover 110 and flows into the device chamber 113, then the front-end side of the sensor element 10 is exposed to the measurement-object gas. Then, when the measurement-object gas passes through the porous protective layer 30 to reach the outside electrode 24, and arrives in the sensor element 10 through the gas introduction port 21, the detection unit 23 generates an electrical signal (the pump current Ip2 herein) according to NOx concentration in the measurement-object gas as described above. Then, based on the electrical signal, for instance, a control unit (not illustrated) electrically connected to sensor element 10 detects the NOx concentration in the measurement-object gas.

At this point, water may be contained in the measurement-object gas, and the water may be condensed in the device chamber 113, water which has been condensed in the pipe 140 may enter the device chamber 113, and the water (water droplet) may adhere to the element body 20. Here, the heater 29 intensively heats the front end and its vicinity of the element body 20, where the electrodes of the detection unit 23 are present. Therefore, the temperature at a portion of the element body 20 is relatively lower than the temperature at the front end and its vicinity of the element body 20, the portion being rearward of the porous protective layer 30, not covered by the porous protective layer 30, and exposed to the device chamber 113. Thus, water droplet is relatively more likely to adhere to the portion of the element body 20, exposed to the device chamber 113 than to the surface of the porous protective layer 30. Also, with the gas sensor 100 mounted on the pipe 140, the longitudinal direction of the sensor element 10 may be parallel to the vertical direction, or inclined approximately 45° with respect to the vertical direction, and thus the front end of the element body 20 is often positioned lower than the rear end. For this reason, the water droplet adhering to the element body 20 is likely to move forward along the surface of the element body 20. Consequently, the water, which has moved along the surface of the element body 20, may adhere to the rear end face 31a of the porous protective layer 30.

However, in the sensor element 10 of the present embodiment, the surface area S of the rear end face 31a of the first protective layer 31 that covers the outside electrode 24 is greater than or equal to 0.9 mm². Since the surface area S is large like this, when the water, which has moved forward along the surface of the element body 20, adheres to the rear end face 31a, the water is likely to spread in a surface direction on the rear end face 31a. Therefore, it is possible to inhibit the water adhering to the rear end face 31a from reaching the outside electrode 24 through the inside of the first protective layer 31. Consequently, it is possible to inhibit degradation of the outside electrode 24 due to poisonous substances (for instance, P, Si, S and the like) in a measurement-object gas, contained in the water, and to inhibit the occurrence of abnormality, such as reduction in accuracy of detection of a specific gas concentration by the sensor element 10.

A larger surface area S makes it possible to further inhibit the water adhering to the rear end face 31a from reaching the outside electrode 24. For instance, the surface area S is preferably 1.0 mm² or greater, more preferably 1.5 mm² or greater, still more preferably 2.2 mm² or greater, still further preferably 2.5 mm² or greater, and still further more preferably 3.0 mm² or greater. The surface area S may be, for instance, 4.0 mm² or less.

As described above, it is preferable that the minimum distance D between the outside electrode 24 and the rear end face 31a be greater than or equal to 2 mm. Since the minimum distance D is large like this, the outside electrode 24 is present at a position away from the contact portion between the rear end face 31a and the first face 20a, in other words, the portion at which water moving forward along the first face 20a of the element body 20 reaches the rear end face 31a for the first time. Thus, with the minimum distance D greater than or equal to 2 mm, it is possible to further inhibit the water adhering to the rear end face 31a from reaching the outside electrode 24 through the inside of the first protective layer 31. From this viewpoint, the minimum distance D is more preferably 4 mm or greater, still more preferably 5 mm or greater, and still further preferably 6 mm or greater. The minimum distance D may be 10 mm or less, or may be 8 mm or less.

As described above, it is preferable that the thickness T of the first protective layer 31 be 0.03 mm or greater and 1 mm or less. With the thickness T greater than or equal to 0.03 am, it is possible to inhibit the water adhering to the upper surface of the first protective layer 31 and containing poisonous substances from moving through the inside of the first protective layer 31 in the thickness direction (the downward direction herein) and reaching the outside electrode 24. From this viewpoint, the thickness T is more preferably 0.1 mm or greater, still more preferably 0.2 mm or greater, and still further preferably 0.3 mm or greater. In addition, with the thickness T greater than or equal to 0.2 am, it is possible to reduce thermal shock to the element body 20 when water adheres to the upper surface of the first protective layer 31, and the effect of improving the water resistance of the element body 20 is also obtained. In addition, with the thickness T less than or equal to 1 mm, it is possible to inhibit the reduction in the responsiveness of detection of a specific gas concentration by the sensor element 10. From this viewpoint, the thickness T is more preferably 0.8 mm or less, and still more preferably 0.5 mm or less.

Also, the inclination angle θ of the above-described rear end face 31a may be 10° or greater and 90° or less as described above. In the present embodiment, the inclination angle θ is 10° or greater and less than 90°. With the inclination angle θ greater than or equal to 10°, the first protective layer 31 is easily manufactured. With the inclination angle θ less than 90°, the surface area S is likely to be increased, as compared with when the inclination angle θ is 90°. For instance, even with the same thickness T, when the inclination angle θ is less than 90°, the surface area S can be increased. The inclination angle θ may be less than or equal to 80°. The inclination angle θ may be 40° or greater and 50° or less.

One or more numerical ranges of the above-described degree of porosity, arithmetic average roughness Ra, and thickness T of the first protective layer 31 may be applied to each of the second to fifth protective layers 32 to 35. One or more numerical ranges of the above-described surface area S and inclination angle θ of the first protective layer 31 may be applied to the second protective layer 32. Although the thickness of each of the second to fifth protective layers 32 to 35 is the same as the thickness T of the first protective layer 31 in the present embodiment, the thickness may differ from each other. In the present embodiment, the surface area S of the second protective layer 32 is greater than or equal to 0.9 mm², the inclination angle θ is 10° or greater and less than 90°, and the first protective layer 31 and the second protective layer 32 have the same surface area S and inclination angle θ. However, one or more of the surface area S and the inclination angle θ may be different.

With the gas sensor 100 of the present embodiment described in detail above, since the surface area S of the rear end face 31a of the first protective layer 31 of the sensor element 10 is greater than or equal to 0.9 mm², it is possible to inhibit the water adhering to the rear end face 31a and containing poisonous substances from reaching the outside electrode 24 through the inside of the first protective layer 31. In addition, with the minimun distance D greater than or equal to 2 mm, it is possible to further inhibit the water adhering to the rear end face 31a from reaching the outside electrode 24 through the inside of the first protective layer 31. Furthermore, with the thickness T of the first protective layer 31 greater than or equal to 0.03 mm, it is possible to inhibit the water containing poisonous substances from moving in the thickness direction (the downward direction herein) from the upper surface of the first protective layer 31 and reaching the outside electrode 24. With the thickness T less than or equal to 1 mm, it is possible to inhibit the reduction in the responsiveness of detection of a specific gas concentration by the sensor element 10.

The rear end face 31a of the first protective layer 31 has a shape curved to be recessed deeper at the center between the right and left. Thus, the surface area S is likely to be increased, as compared with when the rear end face 31a has a planar shape without a curve, for instance. For instance, even with the same thickness T and inclination angle θ, the surface area S can be increased.

It is to be noted that the present invention is not limited to the above-described embodiment, and needless to say, the invention may be implemented in various aspects within the technical scope of the present invention.

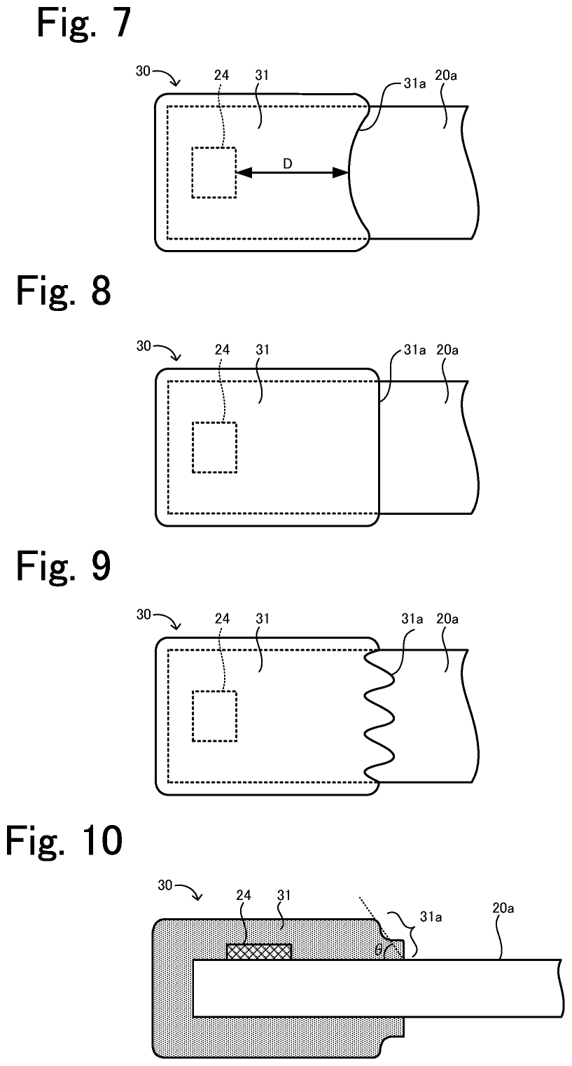

For instance, in the embodiment described above, the inclination angle θ is less than 90°. However, the inclination angle θ may be 90°. For instance, the rear end face 31a in the modification illustrated in FIG. 7 may be used. In FIG. 7, the rear end face 31a has a shape curved to be recessed deeper at the center between the right and left, and the inclination angle θ is 90°.

In the embodiment described above, the rear end face 31a has a shape curved to be recessed deeper at the center between the right and left, however, this is not always the case. For instance, the rear end face 31a in the modification illustrated in FIGS. 8 to 10 may be used. In FIG. 8, the rear end face 31a has a planar shape without a curve. In FIG. 9, the rear end face 31a has a plurality of recessed portions which are recessed forward. In FIG. 10, the rear end face 31a has a shape with a vertical level difference. Even with the shape of the rear end face 31a as in FIGS. 9, 10, the surface area S is likely to be increased. Although the inclination angle θ is 90° in each of FIGS. 8, 9, the inclination angle θ may be changed. Even with the shape of the rear end face 31a illustrated in FIG. 10, the inclination angle θ can be derived as an average value by the calculation method described above (see FIG. 10).

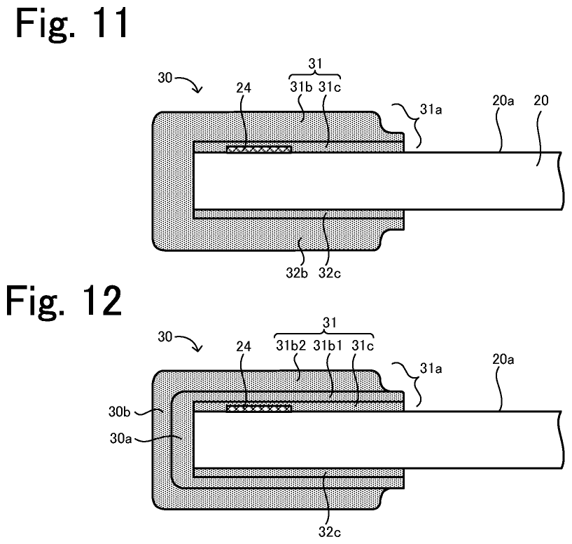

In the embodiment described above, the porous protective layer 30 may include a plurality of layers stacked in the thick direction. For instance, as illustrated in FIG. 11, the porous protective layer 30 may include layers 31c, 32c that cover the first and second faces 20a, 20b; and layers 31b, 32b that further cover the layers 31c, 32c. In this case, similarly to the embodiment described above, the portion (the layers 31b, 31c herein), present immediately above the first face 20a, of the porous protective layer 30 is defined as the first protective layer 31. Thus, the rear end faces of the layers 31b, 31c form the rear end face 31a. As illustrated in FIG. 12, the porous protective layer 30 may have a lower layer 30a closer to the element body 20, and an upper layer 30b away from the element body 20. Also in this case, similarly to the embodiment described above, the portion (the layers 31b1, 31b2, 31c herein), present immediately above the first face 20a, of the porous protective layer 30 is defined as the first protective layer 31. Thus, the rear end faces of the layers 31b1, 31b2, 31c form the rear end face 31a. In the example of FIGS. 11, 12, the outside electrode 24 is covered by the layer 31c. In FIG. 12, the layers 31c, 32c may be omitted. Also, the layer 31b and the layer 31c in FIG. 11 may have different characteristics (for instance, the degree of porosity and the particle diameter of constituent particles) from each other. Similarly, the layers 31b1, 31b2, 31c of FIG. 12 may have different characteristics from each other.

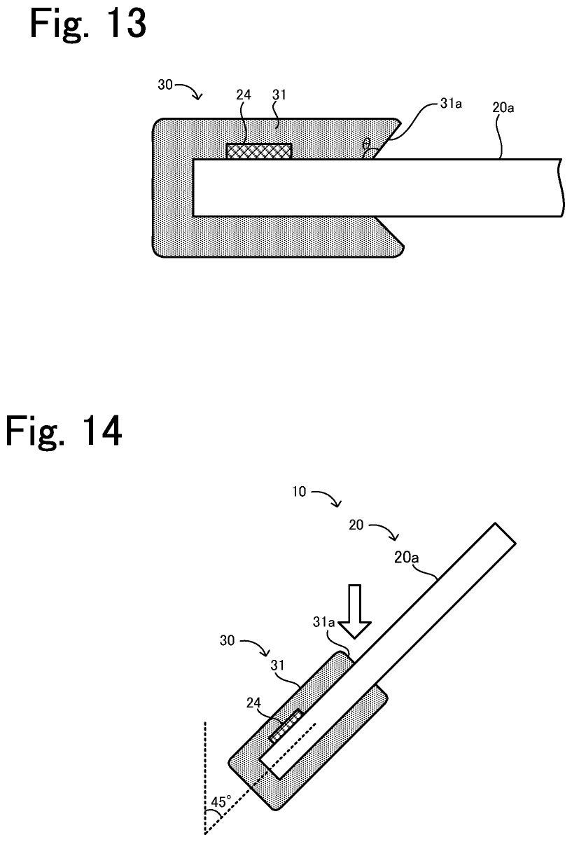

In the embodiment described above, the range of 10° or greater and 90° or less has been illustrated as the numerical range of the inclination angle θ. However, this is not always the case, and the inclination angle θ may be 10° or greater and 170° or less. With the inclination angle θ less than or equal to 170°, the first protective layer 31 is easily manufactured, as compared with when the inclination angle θ is greater than 170°. For instance, as in the rear end face 31a illustrated in FIG. 13, the inclination angle θ may be greater than 90° and 170° or less. With the inclination angle θ greater than 90°, the surface area S is likely to be increased, as compared with when the inclination angle θ is 90°. The inclination angle θ may be greater than or equal to 100°. The inclination angle θ may be 130° or greater and 140° or less. The inclination angle θ may be any value in the range of 10° or greater and 80° or less, and 100° or greater and 170° or less.

In the embodiment described above, the thickness T of the first protective layer 31 is set to the average value measured by using three cross sections, however, the thickness of the first protective layer 31 may be substantially the same at any position. For instance, the thickness of the first protective layer 31 may fall within 0.9 T or greater and 1.1 T or less, or within 0.95 T or greater and 1.05 T or less at any position. “The thickness of the first protective layer 31” herein refers to the thickness of the portion of the first protective layer 31, excluding the end potion and the end face such as the rear end face 31a.

In the embodiment described above, the porous protective layer 30 includes the first to fifth protective layers 31 to 35, however, this is not always the case. It is sufficient that the porous protective layer 30 include the first protective layer 31 that covers at least the outside electrode 24.

In the embodiment described above, the element body 20 of the sensor element 10 has a laminated body of a plurality of (six in FIG. 3) solid electrolyte layers. However, this is not always the case. It is sufficient that the element body 20 include at least one oxygen-ion-conductive solid electrolyte layer. For instance, five layers other than the uppermost solid electrolyte layer in FIG. 3 may be layers (for instance, layers composed of alumina) composed of a material other than the solid electrolyte. In this case, the electrodes of the sensor element 10 may be disposed on the uppermost solid electrolyte layer. For instance, the measurement electrode 27 of FIG. 3 may be disposed on the lower surface of the solid electrolyte layer. Alternatively, the reference gas introduction port 22 may be provided in the second layer from the top, and the reference electrode 28 may be provided rearward of the measurement-object gas distribution portion and on the lower surface of the solid electrolyte layer.

### EXAMPLES

Hereinafter, an instance of specifically manufactured sensor element will be described as an example. Experimental examples 1 to 4 correspond to the examples of the present invention, and experimental example 5 corresponds to a comparative example. It is to be noted that the present invention is not limited to the following examples.

### Experimental Example 1

The sensor element 10 illustrated in FIGS. 1 to 3 was manufactured, which provides experimental example 1. However, the rear end face 31a was manufactured so that the inclination angle θ is 90° as in the rear end face 31a of FIG. 7. Manufacturing of the sensor element 10 in experimental example 1 was conducted in the following manner. First, zirconia particles containing 4 mol % of yttria serving as a stabilizing agent, an organic binder, dispersant, plasticizer, and an organic solvent were mixed, and molded by tape molding to prepare six ceramic green sheets. In the green sheets, a plurality of sheet holes and necessary through holes were formed in advance, the sheet holes being used for positioning at the time of print and lamination. A pattern of a conductive paste for forming the electrodes 24 to 28 of the detection unit 23, including the outside electrode 24, and the heater 29 was printed on each green sheet. Then, the six green sheets are laminated in a predetermined order, and compressed by applying predetermined temperature and pressure conditions. An unfired element body in the size of the element body 20 was cut out from thus obtained compressed body. The cut-out unfired element body was fired to obtain the element body 20. The conductive paste for the outside electrode 24 was produced by mixing Pt particles, zirconia particles, and a binder. Next, the porous protective layer 30 was formed on the surface of the element body 20 by plasma spraying to obtain the sensor element 10. The powder spraying material used for plasma spraying was alumina. The shape of the rear end face 31a of the first protective layer 31 was adjusted using a mask. The dimensions of the element body 20 in experimental example 1 had a length of 67.5 m, a width of 4.25 mm, and a thickness of 1.45 mm. The first protective layer 31 had a degree of porosity of 20%, and a thickness T of 0.2 μm. The rear end face 31a of the first protective layer 31 had a surface area S of 1.8 mm², and an inclination angle θ of 90°. The minimum distance D between the outside electrode 24 and the rear end face 31a was 6 mm. The rear end face 31a of the first protective layer 31 had an arithmetic average roughness Ra of 10 μm.

### Experimental Example 2

The shape of the mask was changed so that the rear end face 31a had a planar shape without a curve, and the sensor element 10 of experimental example 2 was produced similarly to experimental example 1 except that the thickness T was changed. In experimental example 2, the thickness T was 0.3 μm, the surface area S was 1.7 mm², and the inclination angle θ was 50°. The minimum distance D was 7 mm.

### Experimental Examples 3 to 5

The rear end face 31a had still a planar shape without a curve, and the sensor elements 10 of experimental examples 3 to 5 were produced similarly to experimental example 2 except that the surface area S, the thickness T, and the inclination angle θ were variously changed. In experimental example 3, the thickness T was 0.3 μm, the surface area S was 1.3 mm², and the inclination angle θ was 90°. In experimental example 4, the thickness T was 0.2 μm, the surface area S was 0.9 mm², and the inclination angle θ was 90°. In experimental example 5, the thickness T was 0.1 μm, the surface area S was 0.4 mm², and the inclination angle θ was 90°. The minimum distance D in each of experimental examples 3 to 5 was 7 mm.

[Evaluation of Poisoning Resistance]

A poisoning resistance test for evaluating the poisoning resistance of the outside electrode 24 was conducted for the sensor elements 10 of experimental examples 1 to 5. Specifically, first, the vicinity of the detection unit 23 of the sensor element 10 was maintained at 100° C. by the heater 29 with the sensor element 10 inclined 45° as in FIG. 14. The temperature of the vicinity of the rear end face 31a of the element body 20 in this state was approximately 80° C. In this state, a water solution containing siloxane (octamethylcyclotetrasiloxane) at a concentration of 0.12 cc/L was continuously dropped on the contact portion between the rear end face 31a of the sensor element 10 and the first face 20a at a rate of 2 μL/30 sec (see the white arrow of FIG. 14). Before the start of dropping, and at the timing of every six hours since the start of dropping, the vicinity of the detection unit 23 of the element body 20 was heated to 800° C. by the heater 29, the sensor element 10 was driven, and the output (pump current Ip2) of the sensor element 10 was measured. The change rate of the pump current Ip2 after the start of dropping with respect to the pump current Ip2 before the start of dropping was derived, and when the change rate was in the range of 0% or less and greater than −5%, no problem (OK) was determined. In contrast, when the change rate was −5% or less (the absolute value of the change rate was 5% or greater), abnormality (NG) was determined. The sensor element 10 was placed in atmospheric air during the poisoning resistance test. In other words, the measurement-object gas was atmospheric air (NOx concentration is zero). When the outside electrode 24 is poisoned by siloxane and is degraded, the sensitivity characteristics of the detection unit 23 is changed, thus the pump current Ip2 is changed even though the NOx concentration in the measurement-object gas is not changed. Thus, when the pump current Ip2 is changed even before the start of dropping, this indicates that the dropped water solution in a great amount has reached the outside electrode 24 which has been poisoned. For each of the sensor elements of experimental examples 1 to 5, dropping of the water solution and measurement of the pump current Ip2 every six hours were continued until abnormality was determined. The surface area S, the thickness T, and the inclination angle θ of the sensor elements 10 of experimental examples 1 to 5, and the result of the poisoning resistance test are shown in Table 1.

TABLE 1

| | | | | Poisoning resistance test | | | | |
|---|---|---|---|---|---|---|---|---|
| | | | | | After | After | After | After |
| | | | | After six | twelve | eighteen | twenty four | thirty |
| | | | | hours | hours | hours | hours | hours |
| | Surface | | Inclination | Amout of | Amout of | Amout of | Amout of | Amout of |
| | area S | Thickness | angle θ | dropping | dropping | dropping | dropping | dropping |
| | [mm²] | T [μm] | [°] | 1.44 mL | 2.88 mL | 4.32 mL | 5.76 mL | 7.2 mL |
| Experimental | 1.8 | 0.2 | 90 | OK | OK | OK | OK | NG |
| Example 1 | | | | | | | | |
| Experimental | 1.7 | 0.3 | 50 | OK | OK | OK | OK | NG |
| Example 2 | | | | | | | | |
| Experimental | 1.3 | 0.3 | 90 | OK | OK | OK | NG | |
| Example 3 | | | | | | | | |
| Experimental | 0.9 | 0.2 | 90 | OK | OK | NG | | |
| Example 4 | | | | | | | | |
| Experimental | 0.4 | 0.1 | 90 | NG | | | | |
| Example 5 | | | | | | | | |

As shown in Table 1, for a larger surface area S, even when a long time has elapsed, in other words, even when the amount of dropping is large, the pump current Ip2 is unlikely to change. Thus, it has been verified that for a larger surface area S of the rear end face 31a, it is possible to further inhibit the water containing poisonous substances from reaching the outside electrode 24. In experimental example 5 in which the surface area S was 0.4 mm², abnormality was determined at the time of the first determination (six hours after the start of dropping), whereas in experimental examples 1 to 4 in which the surface area S was greater than or equal to 0.9 mm², abnormality was not determined even at the time of the second determination. From this result, it is probably possible to inhibit the water containing poisonous substances from reaching the outside electrode 24 by setting the surface area S to 0.9 mm² or greater. In addition, from the comparison between experimental examples 3, 4, the surface area S is preferably 1.0 mm² or greater, and from the comparison between experimental examples 1 to 3, the surface area S is more preferably 1.5 mm² or greater.

What is claimed is:

1. A sensor element comprising:
an element body that includes an oxygen-ion-conductive solid electrolyte layer, the element body being in an elongated rectangular parallelepiped shape having a longitudinal direction;
a detection unit that has a plurality of electrodes disposed on a front-end side of the element body, and detects a specific gas concentration in a measurement-object gas, the longitudinal direction being a front-rear direction;
an outside electrode that is one of the plurality of electrodes and disposed on a first face which is a surface along the longitudinal direction of the element body; and
a porous first protective layer that is disposed on the first face and covers the outside electrode,
wherein a surface area S of a rear end face of the first protective layer is greater than or equal to 0.9 mm².

2. The sensor element according to claim 1,
wherein a minimum distance D from a rear end of the outside electrode to a contact portion between the rear end face and the first face is greater than or equal to 2 mm.

3. The sensor element according to claim 1,
wherein a thickness T of the first protective layer is greater than or equal to 0.03 mm and less than or equal to 1 mm.

4. The sensor element according to claim 1,
wherein the rear end face of the first protective layer has a shape curved to be recessed at a center between right and left, and a direction of the right and left is parallel to the first face and perpendicular to the longitudinal direction.

5. The sensor element according to claim 1,
wherein the rear end face of the first protective layer has an inclination angle θ greater than or equal to 10° and less than or equal to 90° with respect to the first face.

6. A gas sensor comprising the sensor element according to claim 1.
